(19) United States
(12) Patent Application Publication
(10) Pub. No.: US 2018/0344219 A1
(43) Pub. Date: Dec. 6, 2018

(54) MULTI COLOR PULSE OXIMETER

(71) Applicant: KONINKLIJKE PHILIPS N.V., EINDHOVEN (NL)

(72) Inventors: John CRONIN, BONITA SPRINGS, FL (US); Joseph BODKIN, FORT MEYERS, FL (US)

(21) Appl. No.: 15/761,465

(22) PCT Filed: Sep. 21, 2016
(86) PCT No.: PCT/EP2016/072340

§ 371 Date: Mar. 20, 2018

Related U.S. Application Data
(60) Provisional application No. 62/222,529, filed on Sep. 23, 2015.

(30) Foreign Application Priority Data
Mar. 18, 2016 (EP) 16161242.9

(51) Int. Cl. A61B 5/1495; A61B 5/1455; A61B 5/00; G16H 50/30; G16H 40/63; G06F 17/30

(52) U.S. Cl. CPC A61B 5/1495; A61B 5/14552; A61B 5/743; A61B 5/7435; A61B 2560/0271; G16H 50/30; G16H 40/63; G06F 17/30864; A61B 5/0022

(57) ABSTRACT

Disclosed are systems and methods for measuring blood oxygen saturation via a multiwavelength pulse oximeter. The system comprises a multi-wavelength pulse oximeter with an LED array, a patient monitor connected to the multi-wavelength pulse oximeter, a medical data network for querying an at least one wavelength associated with a patient condition, an algorithm network for providing subscription based algorithms to process sensor data, and a user interface on the patient monitor for configuring a pulse oximeter's scanning mode, for querying the at least one wavelength associated with a patient condition, and for displaying a plethysmograph.

### BACKGROUND OF THE INVENTION

Pulse oximeters are spectrophotometric devices that use spectroscopy for monitoring desired physiological characteristics of a patient. Pulse oximeters noninvasively monitor the blood oxygen saturation (SpO2) of a person. As such, a wide variety of pulse oximeters have been developed to provide doctors and other healthcare personnel with the information they need to provide the best possible healthcare for their patients.

Pulse oximeters emit wavelengths of light through the body part to a photodetector to measure the amount of oxygen being carried in the blood. Current pulse oximeters usually use at least two wavelengths of light, infrared and red. Also, although there are certain wavelengths of light that are proven more effective for use in pulse oximetry, current pulse oximeters are designed to emit and detect only a limited number of wavelengths of light. Hence, there is a need for a pulse oximeter that can scan the full electromagnetic spectrum, outputting only the most relevant wavelengths associated to a patient's current condition.

### SUMMARY OF THE CLAIMED INVENTION

Some embodiments of the present invention relates to systems and methods for measuring blood oxygen saturation via a multi-wavelength pulse oximeter. The system comprises a multi-wavelength pulse oximeter with an LED array, a patient monitor connected to the multi-wavelength pulse oximeter, a medical data network for querying an at least one wavelength associated with a patient condition, an algorithm network for providing subscription based algorithms to process sensor data, and a user interface on the patient monitor for configuring a pulse oximeter's scanning mode, for querying the at least one wavelength associated with a patient condition, and for displaying a plethysmograph.

The method of the present invention comprises connecting a patient monitor to a medical data network; entering using the patient monitor at least one wavelength in a search query to the medical data network for a patient condition associated with the at least one wavelength; displaying query results corresponding to at least one wavelength on a user interface of the patient monitor; requesting to scan an initial wavelength via the patient monitor's user interface; transmitting the initial wavelength to be scanned to a sensor controller in the patient monitor; outputting the initial wavelength on the multi-wavelength pulse oximeter via a control signal from the controller; acquiring a first set of pulse oximeter data corresponding to the initial wavelength and storing the acquired first set of pulse oximeter data to a sensor database; incrementing the initial wavelength to a succeeding wavelength; acquiring a second set of pulse oximeter data corresponding to the succeeding wavelength and storing the acquired second set of pulse oximeter data to the sensor database; and diagnosing the patient's condition based on the acquired first and second set of pulse oximeter data.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a block diagram of a system for measuring blood oxygen saturation via a multi-wavelength pulse oximeter according to a preferred embodiment of the present invention.

FIG. 2 illustrates a block diagram of a patient monitor according to a preferred embodiment of the present invention.

FIG. 3 illustrates a flowchart according to a preferred embodiment of the present invention.

FIGS. 4A, 4B, and 4C illustrate the flowcharts in configuring the scanning mode of the multi-wavelength pulse oximeter according to a preferred embodiment of the present invention.

FIGS. 5A, 5B, 5C and 5D illustrate a graphical user interface of the patient monitor according to a preferred embodiment of the present invention.

FIG. 6 illustrates a flowchart for a lookup software according to a preferred embodiment of the present invention.

FIGS. 7A and 7B illustrates the flowcharts for a calibration software and an algorithm Selection software according

### DETAILED DESCRIPTION OF THE EMBODIMENTS

An embodiment of the present invention relates to a method for measuring blood oxygen saturation via a multi-wavelength pulse oximeter comprising: connecting a patient monitor to a medical data network; entering using the patient monitor at least one wavelength in a search query to the medical data network for a patient condition associated with the at least one wavelength; displaying query results corresponding to at least one wavelength on a user interface of the patient monitor; requesting to scan an initial wavelength via the patient monitor's user interface; transmitting the initial wavelength to be scanned to a sensor controller in the patient monitor; outputting the initial wavelength on the multi-wavelength pulse oximeter via a control signal from the controller; acquiring a first set of pulse oximeter data corresponding to the initial wavelength and storing the acquired first set of pulse oximeter data to a sensor database; incrementing the initial wavelength to a succeeding wavelength; acquiring a second set of pulse oximeter data corresponding to the succeeding wavelength and storing the acquired second set of pulse oximeter data to the sensor database; and diagnosing the patient's condition based on the acquired first and second set of pulse oximeter data.

Embodiments of the present invention also relate to a system for measuring blood oxygen saturation via a multi-wavelength pulse oximeter comprising: a multi-wavelength pulse oximeter with an LED array; a patient monitor connected to the multi-wavelength pulse oximeter; a medical data network for querying an at least one wavelength associated with a patient condition; and a user interface on the patient monitor for configuring a pulse oximeter's scanning mode, for querying the at least one wavelength associated with a patient condition, and for displaying a plethysmograph.

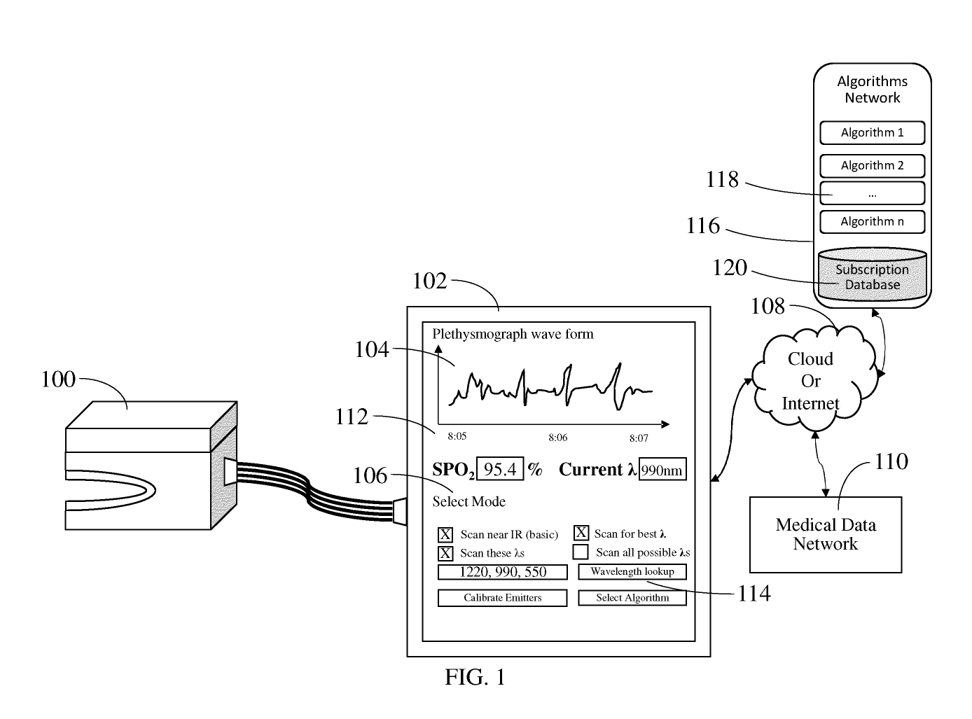

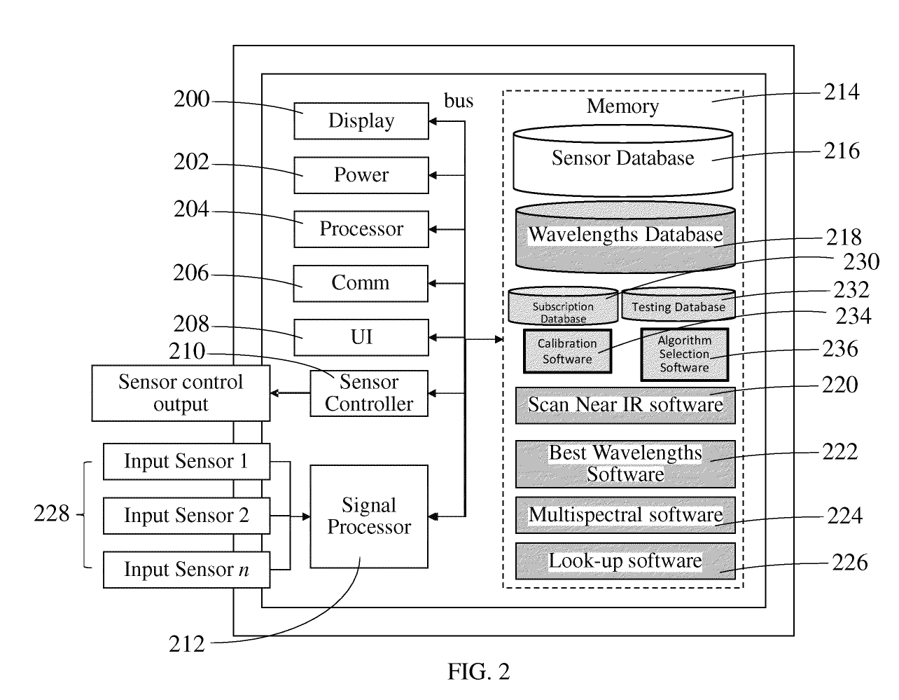

In a preferred embodiment of the present invention as illustrated in FIG. 1, a system for measuring blood oxygen saturation via a multi-wavelength pulse oximeter comprises a multi-wavelength pulse oximeter 100 and a patient monitor 102 with a graphical user interface 112 which is connected to a medical data network 110 and Algorithms Network 116 with algorithms 1-n 118 and a Subscription database 120, all residing in the cloud or internet 108. The multi-wavelength pulse oximeter 100 preferably emits multiple electromagnetic spectrum wavelengths ranging from infrared to ultraviolet. As shown in FIG. 1, the graphical user interface 112 displays the plethysmograph 104 of the acquired pulse oximeter data, the corresponding wavelengths, and the scanning mode 106 selected by the user. The graphical user interface 112 preferably allows a user to select a scanning mode 10 such as: scanning the full electromagnetic spectrum (a full electromagnetic spectrum scan), a specific wavelength range such as infrared (IR) spectrum, a limited set of wavelengths, or wavelengths with the highest signal-to-noise ratio. The graphical user interface 112 also preferably allows a user, which may be a doctor or medical professional, to query selected wavelengths associated with a patient condition using a lookup software 114. The queries are sent to the medical data network 110 residing in the cloud or internet 108. The results of the queries are then displayed on the graphical user interface 112. The graphical user interface 112 also preferably allows a user, which may be a doctor or medical professional, to calibrate emitters of the pulse oximeter to test accuracy, and select a cloud-based algorithm from the Algorithms Network to analyze the pulse oximeter data collected by the pulse oximeter 100. FIG. 2 illustrates a preferred embodiment of a patient monitor. The patient monitor comprises a display 200, a power module 202, a processor 204, a communications module 206, a user interface 208, a sensor controller 210, a signal processor 212, and a memory 214. The memory 214 comprises a sensor database 216, a wavelengths database 218, a Subscription Database 230, and a Testing Database 232. The memory 214 also comprises programs used for scanning the electromagnetic spectrum—which are Calibration Software 234, Algorithm Selection Software 236, scanning near IR software 220, best wavelength software 222, multispectral software 224, and a lookup software 226. The sensor controller 210 controls the emission of wavelengths of one or more sensors 228 communicably connected to the signal processor. The signal processor processes the acquired data from the plurality of sensors 228. The patient monitor 102 preferably allows storing a wavelength or a set of wavelengths on a wavelengths database 218 on its memory 214. A note may also be stored with the wavelength or set of wavelengths. The patient monitor 102 also preferably receives software updates for the multi-wavelength pulse oximeter 100 and the patient monitor 102. Software updates may include firmware updates or algorithm updates for the various scanning modes 106.

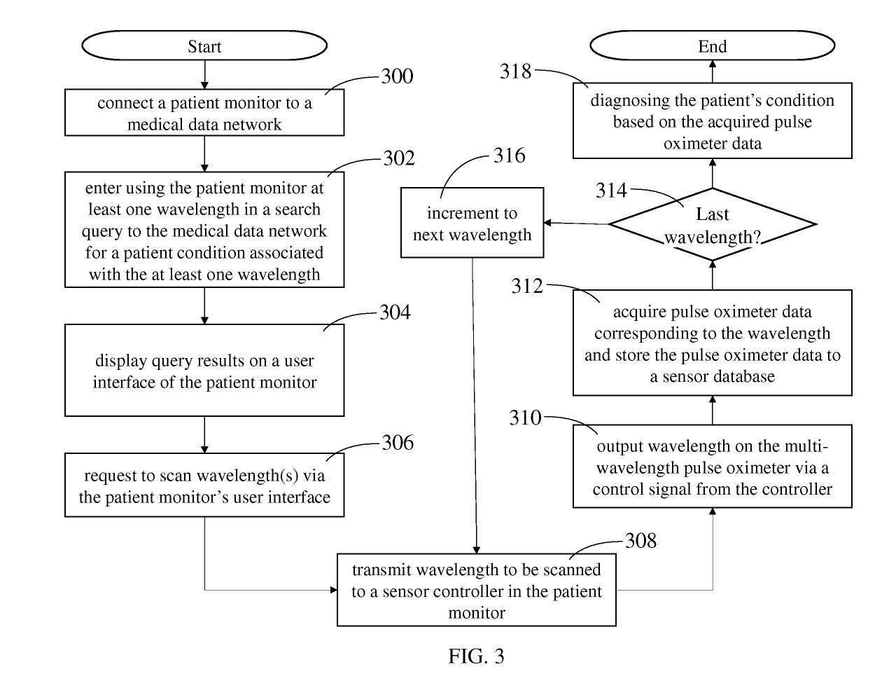

FIG. 3 illustrates a preferred method of the present invention. A patient monitor is connected to a medical data network (step 300). In this embodiment, a medical professional enters in a search query using the patient monitor to the medical data network one or more wavelengths for a patient condition associated with one or more wavelengths (step 302). The results of the search query are displayed on the patient monitor (step 304). The medical professional can then request to scan the one or more wavelengths via the patient monitor's user interface (step 306). The one or more wavelengths are then transmitted to the sensor controller (step 308) and a control signal is then generated to output the corresponding wavelength on the multi-wavelength pulse oximeter (step 310). Pulse oximeter data is then acquired and stored to a sensor database (step 312). If there is still a succeeding wavelength (step 314), the current wavelength is incremented (step 316) and pulse oximeter data is also acquired and stored for that succeeding wavelength. If there are no more succeeding wavelengths, the condition of the patient can then be diagnosed based on the acquired pulse oximeter data (step 318).

In another preferred method of the present invention, the request to scan the one or more wavelengths may include a scanning mode. Scanning modes may be scanning the full electromagnetic spectrum, scanning near the infrared (IR) spectrum, scanning a limited set of wavelengths, or scanning for the wavelengths with the highest signal-to-noise ratio.

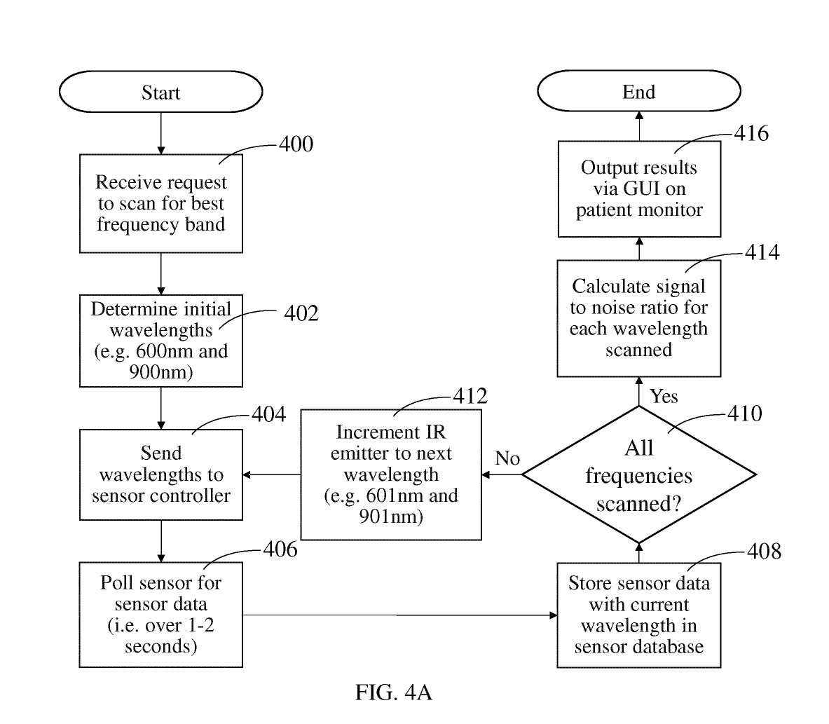

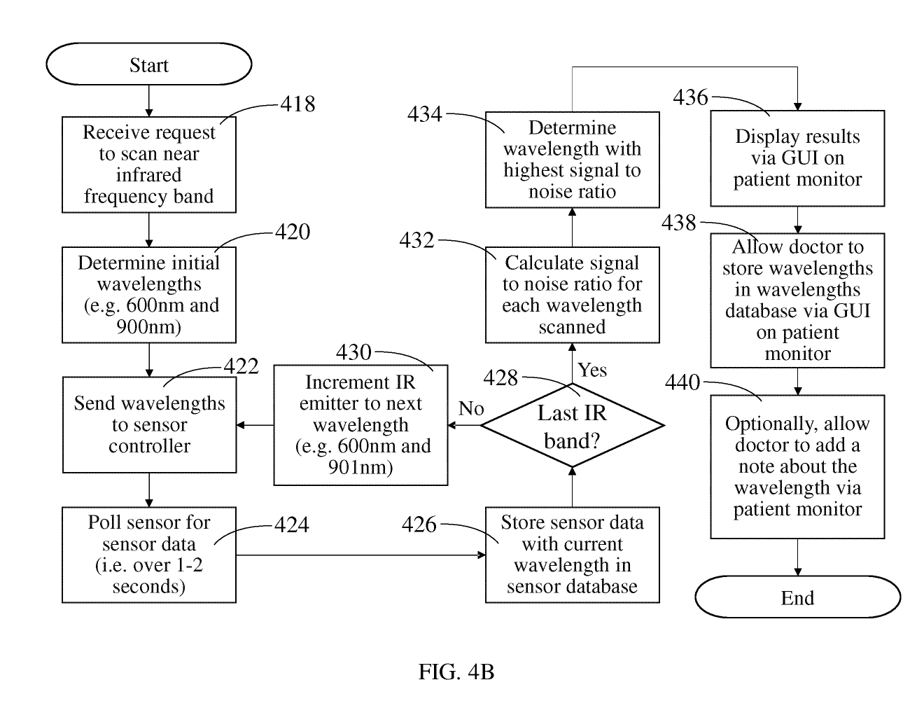

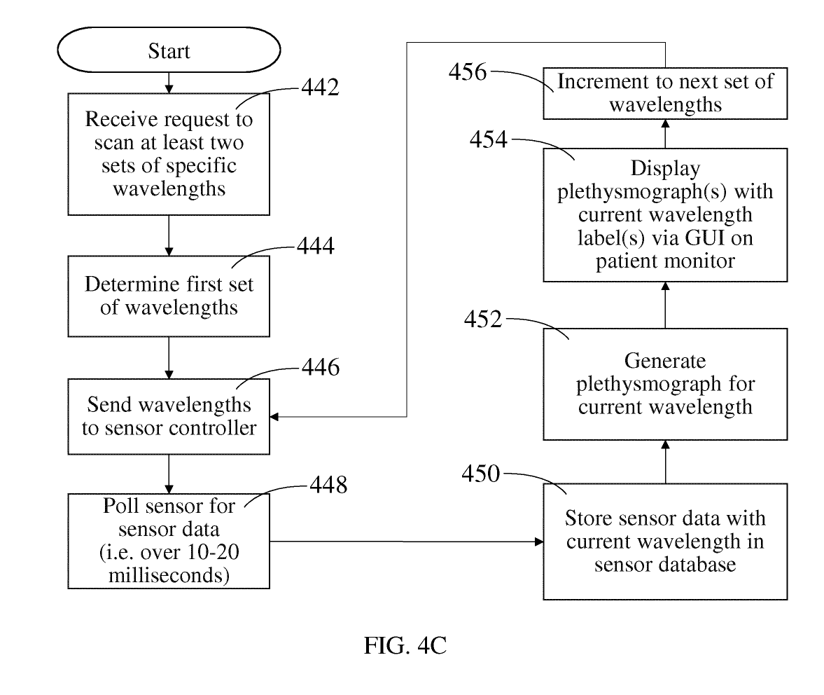

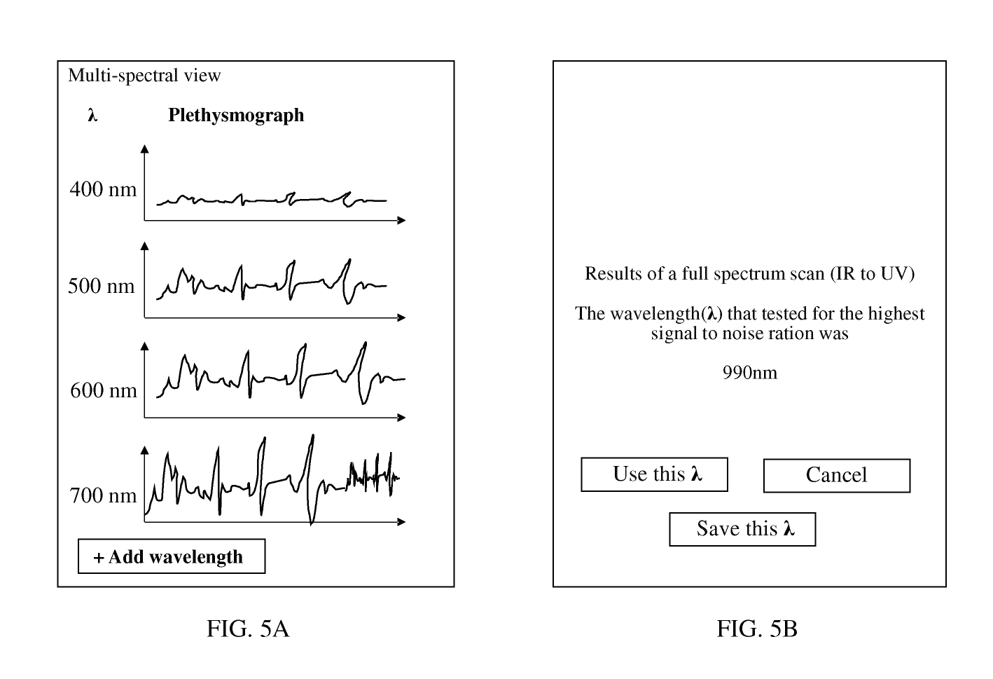

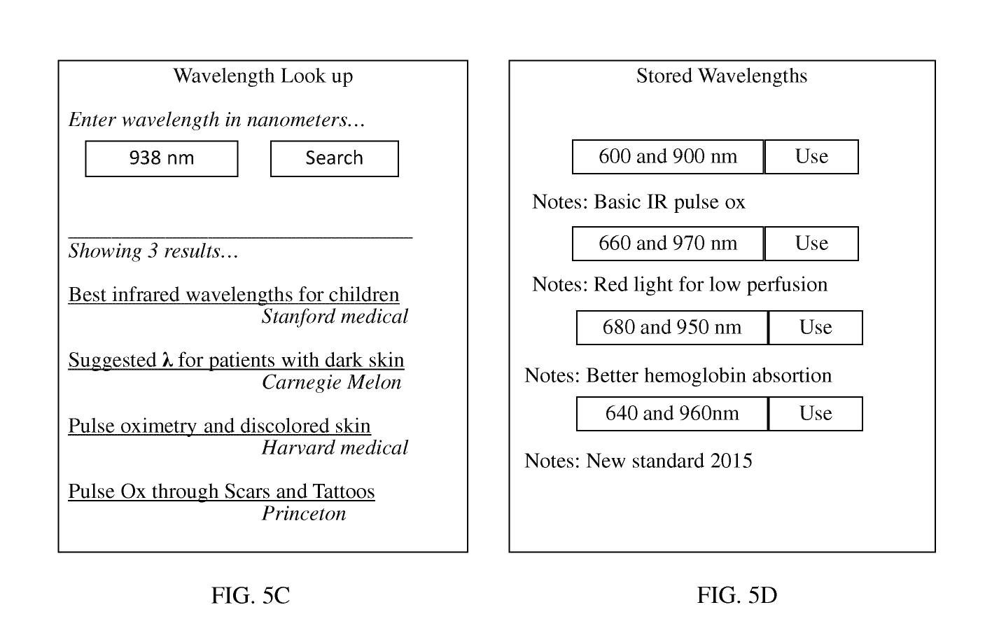

FIG. 4A illustrates the flowchart of a preferred scanning mode of the present invention. The patient monitor receives a request to scan the whole electromagnetic spectrum to determine the wavelengths with the highest signal-to-noise ratio (step 400). The initial wavelength is then determined (step 402) and sent to the sensor controller (step 404). Pulse oximeter data is then acquired for given sample time (step 406) and then stored with the corresponding wavelength in a database (step 408). If there is still a succeeding wavelength (step 410), the current wavelength is incremented (step 412) and pulse oximeter data is also acquired and stored for that succeeding wavelength. After the whole spectrum is scanned, the signal-to-noise ratio for each wavelength is calculated (step 414) and the wavelength with the highest signal-to-noise ratio is displayed on the graphical user interface (step 416) as shown in FIG. 5B.

FIG. 4B illustrates the flowchart of another preferred scanning mode of the present invention. The patient monitor receives a request to scan near the infrared spectrum (418). The initial wavelength in the infrared spectrum is then determined (step 420) and sent to the sensor controller (step 422). Pulse oximeter data is then acquired for a given sample time (step 424) and then stored with the corresponding wavelength in a database (step 426). If there is still a succeeding wavelength in the infrared spectrum (step 428), the current wavelength is incremented (step 430) and pulse oximeter data is also acquired and stored for that succeeding wavelength. After the whole infrared spectrum is scanned, the signal-to-noise ratio for each wavelength is calculated (step 432) and the wavelength with the highest signal-to-noise ratio is determined (step 434) and displayed on the graphical user interface (step 436) as shown in FIG. 5B. Further, the wavelength with the highest signal-to-noise ratio can be stored in a database (step 438). The medical professional may also add notes regarding the wavelength (step 440) as shown in FIG. 5D.

FIG. 4C illustrates the flowchart of another preferred scanning mode of the present invention. The patient monitor receives a request to scan at least two sets of wavelengths (step 442). The first set of wavelengths is determined (step 444) and sent to the sensor controller (step 446). Pulse oximeter data is then acquired for a given sample time (step 448) and then stored with the corresponding wavelength in a database (step 450). A plethysmograph is then generated for the wavelength (step 452) and displayed on the patient monitor (step 454) as shown in FIG. 5A. If there are succeeding sets of wavelengths, the current set is incremented to the next set of wavelengths (step 456) and sent to the sensor controller for pulse oximeter data acquisition.

FIG. 5A illustrates a preferred embodiment of a graphical user interface providing a multi-spectral view in the patient monitor. A single sensor may not be able to monitor multiple spectra simultaneously, but can scan selected wavelengths for a specific sample rate (i.e., 10-20 milliseconds). For example, sample 1 is measured at 400 nm, sample 2 is 500 nm, sample 3 is 600 nm, sample 4 is 700 nm, and sample 5 is 400 again, and so on.

FIG. 5B illustrates another preferred embodiment of a graphical user interface of the patient monitor. The graphical user interface shows the results of a full spectrum scan to determine wavelength with the highest signal to noise ratio. The interface also allows the user to use that wavelength or store it for later use.

FIG. 5C illustrates still another preferred embodiment of a graphical user interface of the patient monitor. The graphical user interface displays the results of a query to the medical data network. For example, the doctor has searched for scholarly articles that contain the wavelength 938 nm to determine if there is any relevant literature relating to this wavelength. Relevant literature maybe related to use of the wavelength on discolored skin, through scars and tattoos, etc.

Another preferred embodiment of a graphical user interface of the patient monitor is illustrated in FIG. 5D. The graphical user interface displays the stored wavelengths that can be used for future monitoring. The graphical user interface also allows a user to make a note for each wavelength stored.

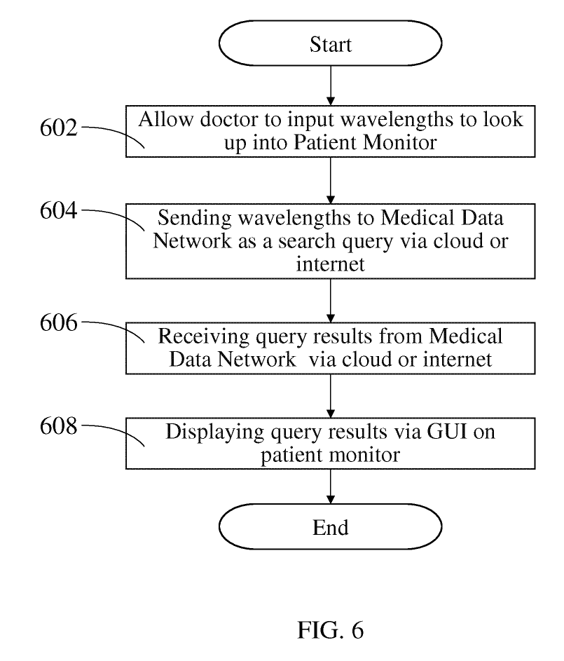

FIG. 6 illustrates the flowchart of a lookup software according to a preferred embodiment of the present invention. A medical professional inputs at least one wavelength as a search query to a patient monitor using the user interface (step 602). The inputted wavelength is then looked up on the medical data network (step 604). The results of the search query are then acquired from the medical data network (step 606) and then displayed on the graphical user interface of the patient monitor (step 608).

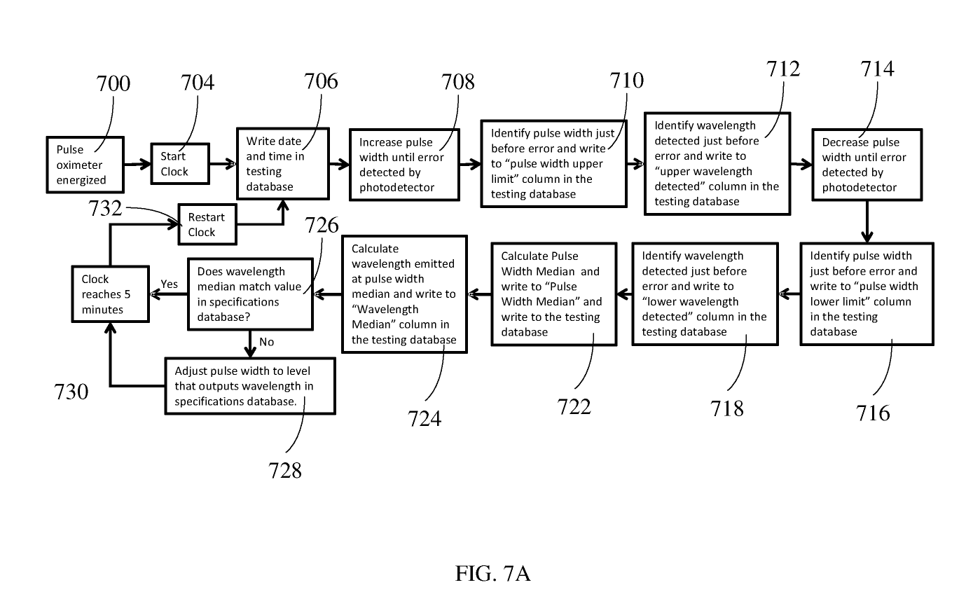

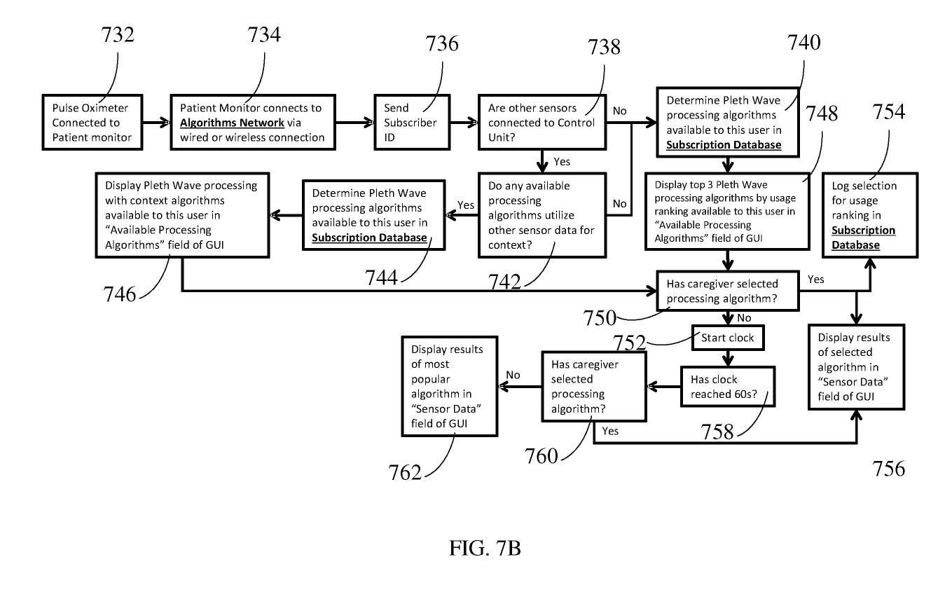

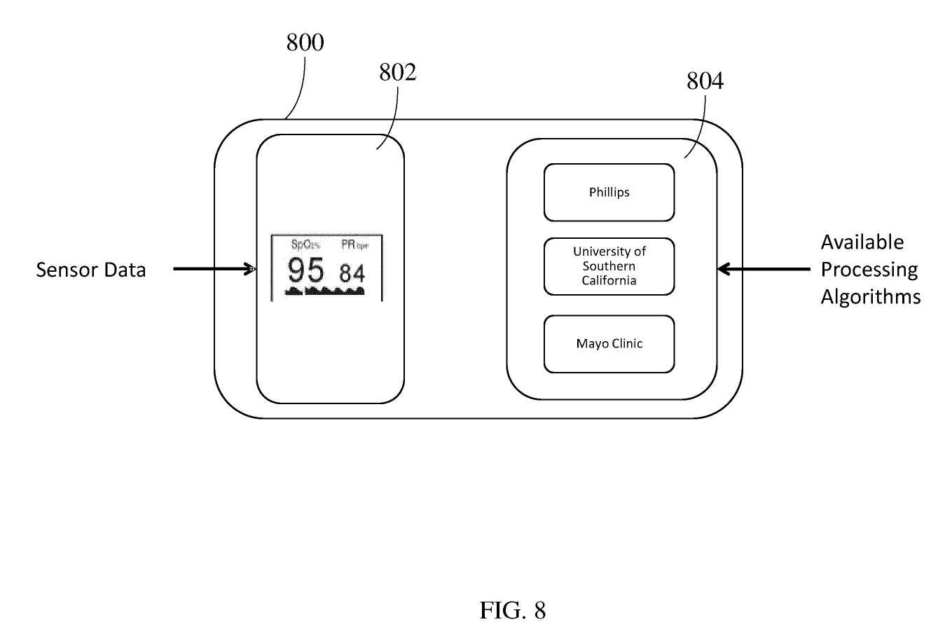

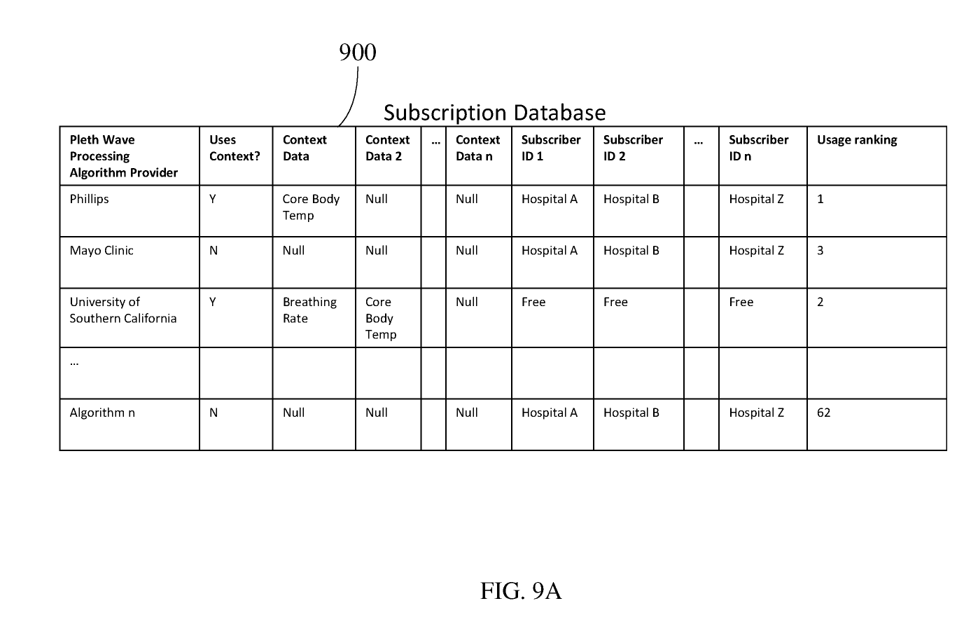

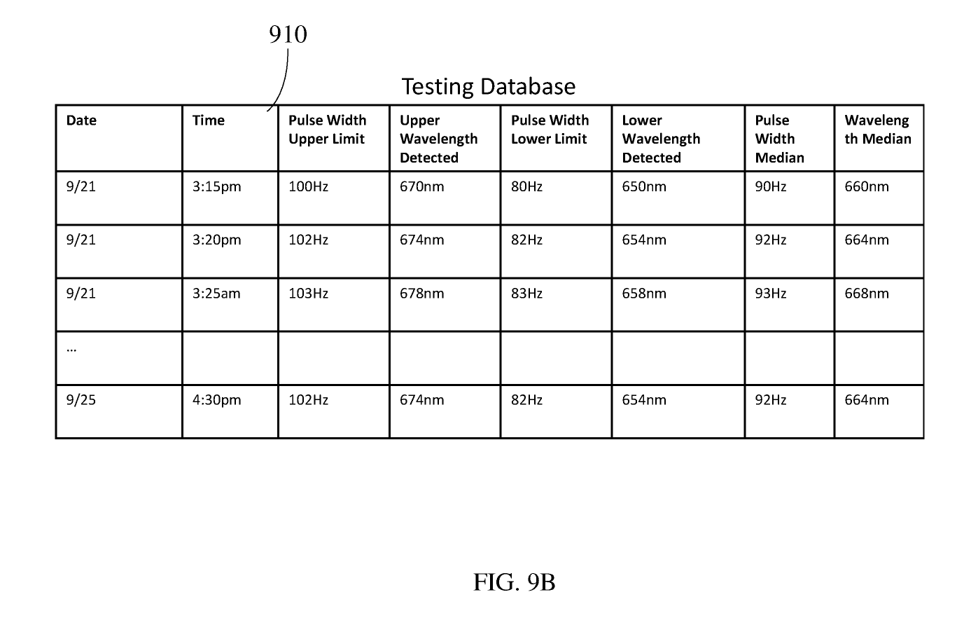

FIG. 7A illustrates the flowchart of a preferred embodiment of a calibration software. Once the pulse oximeter is energized (step 700), the software starts the clock (step 704). The date and time are then recorded in the testing database (step 706). Next, the software sends instructions to the sensor controller 210 to increase the pulse width of the sensor emitters until an error is detected by the sensor (step 708). The software then records the pulse width at which the error occurred as the upper wavelength detected (step 710). The software then repeats the process (step 712, 714, 716), except the pulse width is decreased instead of increased, and the resultant pulse width is recorded as the lower wavelength detected (step 718). A median value is then calculated (step 722) and compared with a standard to determine the error tolerance of the emitters/detectors (step 724, 726, 728). After 5 minutes the process restarts (step 730, 702).

FIG. 7B illustrates the flowchart of an algorithm selection software. Once the pulse oximeter is connected to the patient monitor (step 732), the patient monitor connects to algorithms network over a wired or wireless connection (step 734). The patient monitor sends a subscriber ID (step 736) to the algorithms network and then determines if there are other sensors connected to the patient monitor (step 738), in order to recommend algorithms that incorporate those other sensors (step 742, 744, 746). The software then displays algorithms available to the user based on their sensors and subscription (step 740, 748), and allows the user to select one algorithm to use to process sensor data (step 750, 752, 754, 756, 758, 760, 762). The algorithm maybe related to use of a wavelength on discolored skin, through scars and tattoos, etc.

FIG. 8 illustrates a preferred embodiment of a graphical user interface 800 of a patient monitor. On the left, there is included a display of sensor data 802 collected from the patient monitor's sensors. On the right, there is displayed the available algorithms 804 to analyze sensor data, which the user can select.

FIG. 9A illustrates a preferred embodiment of a subscription database 900. The subscription database 900 contains data about the patient monitors subscription to the algorithm network and the algorithms available to the user.

FIG. 9B illustrates a preferred embodiment of a testing database 910. The testing database 910 contains data generated by the calibration software about the date, time, and results of a sensor calibration.

In an exemplary embodiment of the invention, a doctor wants to know if a 938 nm wavelength is useful in monitoring a certain condition of a patient. The doctor queries the 938 nm wavelength on the medical network and the results of the query are displayed on the patient monitor. After reading the results, the doctor concludes that the 938 nm wavelength is helpful and configures the multi-wavelength pulse oximeter using the patient monitor to scan a selected spectrum around the 938 nm range. One by one, the wavelengths are scanned for a sample time of 10-20 milliseconds, and the corresponding pulse oximeter data are stored in the sensor database. After the selected spectrum is scanned, the doctor can make a diagnosis based on the acquired pulse oximeter data from the set of wavelengths.

In another exemplary embodiment of the invention, a doctor wants to know what the most accurate wavelength is helpful in monitoring the condition of a patient. The doctor requests using the patient monitor to scan the whole electromagnetic spectrum to determine the most accurate wavelength for monitoring a specific condition of a patient. The initial wavelength is transmitted to the sensor controller to control the emitted wavelength of the LED array in a multi-wavelength pulse oximeter. Corresponding pulse oximeter data is then acquired and stored in a sensor database. The initial wavelength is then incremented until it reaches the end of the electromagnetic spectrum with the corresponding pulse oximeter data for each wavelength stored in a sensor database. The signal-to-noise ratio is then calculated for each wavelength and the one with the highest signal-to-noise ratio, for example, 990 nm is displayed. The doctor can now choose to use the 990 nm wavelength now or he can store it to a wavelength database for future use. Additionally, the doctor can also add a note regarding the 990 nm wavelength.

In another exemplary embodiment of the invention, a doctor wants to know if the wavelength displayed on the patient monitor is accurate. The doctor selects calibrate emitters and the calibration software is executed in order to determine if the upper and lower tolerances are compared to a standard.

In another exemplary embodiment of the invention, a doctor wants to select a new algorithm to process the sensor data. The algorithm selection software is executed to synchronize the patient monitor with the algorithm network using subscription data from the subscription database, and allows the doctor to select at least one algorithm from a plurality of algorithms that process sensor data.

In accordance with the various embodiments of the present invention, the memory 214 may include high-speed random access memory or non-volatile memory such as magnetic disk storage devices, optical storage devices, or flash memory. Memory 214 may also store software instructions for facilitating processes, features and applications of the system disclosed in the invention.

In accordance with the various embodiments of the present invention, communications module 206 may include any transmitter or receiver used for Wi-Fi, Bluetooth, infrared, NFC, radio frequency, cellular communication, visible light communication, Li-Fi, WiMax, ZigBee, fiber optic and other forms of wireless communication devices. Alternatively, the communications module 206 may be a physical channel such as a USB cable or other wired forms of communication.

The present invention is not intended to be restricted to the several exemplary embodiments of the invention described above. Other variations that may be envisioned by those skilled in the art are intended to fall within the disclosure.

1. A system for a user to measure blood oxygen saturation via a multi-wavelength pulse oximeter, the system comprising:
a multi-wavelength pulse oximeter with an LED array for acquiring a pulse oximeter data;
a patient monitor connected to the multi-wavelength pulse oximeter, the patient monitor having a graphical user interface for configuring a pulse oximeter's scanning mode, for querying at least one wavelength associated with a patient condition, and for displaying a plethysmograph;
a medical data network for querying an the at least one wavelength associated with the patient condition, the medical data network connected to the patient monitor and residing in the cloud or internet; and
an algorithm network connected to the patient monitor and residing in the cloud or internet, the algorithm network comprising algorithms 1-n and a subscription data base;
wherein the graphical user interface displays the plethysmograph of the acquired pulse oximeter data, one or more corresponding wavelengths, and a scanning mode selected by the user; and
wherein the graphical user interface allows the user to calibrate one or more emitters of the pulse oximeter, and to select at least one of the algorithms 1-n from the algorithms network to analyze the pulse oximeter data collected by the pulse oximeter.

2. The system of claim 1, wherein the multi-wavelength pulse oximeter emits multiple electromagnetic spectrum wavelengths ranging from infrared to ultraviolet.

3. (canceled)

4. The system of claim 1, wherein the graphical user interface allows the user to select a scanning mode selected from a full electromagnetic spectrum scan, a specific wavelength range, a limited set of wavelengths, and wavelengths with the highest signal-to-noise ratio.

5. The system of claim 1, wherein the graphical user interface allows the user to query a selected wavelength associated with a patient condition using a lookup software.

6. (canceled)

7. The system of claim 1, wherein the patient monitor comprises a display, a power module, a processor, a communications module, a user interface, a sensor controller, a signal processor, and a memory.

8. The system of claim 7, wherein the memory storing thereon a sensor database, a wavelengths database, the subscription database, a testing database, and one or more programs for scanning an electromagnetic spectrum.

9. The system of claim 8, wherein the programs for scanning an electromagnetic spectrum is a calibration software, an algorithm selection software, a scanning near IR software, a best wavelength software, a multispectral software, a lookup software, or a combination thereof.

10. The system of claim 7, wherein the sensor controller controls an emission of wavelengths of one or more sensors communicably connected to the signal processor.

11. The system of claim 10, wherein the signal processor acquires and processes sensor data from the sensors.

12. The system of claim 1, wherein the graphical user interface allows the user to make a note for each wavelength stored.

13. The system of claim 1, wherein the graphical user interface provides as multispectral view in the patient monitor.

14. The system of claim 4, wherein, when the full electromagnetic spectrum scan has been selected and performed, the graphical user interface shows a result of the full electromagnetic spectrum scan to determine the wavelength with the highest signal to noise ratio.

15. The system of claim 14, wherein the graphical user interface allows the user to use the determined wavelength or store the determined wavelength for later use.

16. The system of claim 1, wherein the graphical user interface displays at least one of:
a result of a query to the medical data network;
the stored wavelength that can be used for a future monitoring; and
the sensor data and the algorithms 1-n which can be selected by the user.

17. (canceled)

18. (canceled)

19. The system of claim 1, wherein the subscription database contains data about a subscription of the patient monitor to the algorithm network and the algorithms available to the user.

20. The system of claim 9, wherein the testing database contains data generated by the calibration software about a date, a time, and a result of a sensor calibration.

21. (canceled)

22. (canceled)
